(19) United States
(12) Patent Application Publication
(10) Pub. No.: US 2003/0144579 A1
(43) Pub. Date: Jul. 31, 2003

(54) System and method for transmitting vital health statistics to a remote location from a mobile platform

(76) Inventor: Gerald Lee Buss, Newcastle, WA (US)

Correspondence Address: HARNESS, DICKEY & PIERCE, P.L.C., P.O. BOX 828, BLOOMFIELD HILLS, MI, 48303, US

(21) Appl. No.: 10/350,868

(22) Filed: Jan. 23, 2003

Related U.S. Application Data
(63) Continuation-in-part of application No. 09/952,612, filed on Sep. 13, 2001, now abandoned.

(51) Int. Cl. A61B 5/00

(52) U.S. Cl. 600/300

(57) ABSTRACT

A system and method for transmitting vital health statistics from a patient to a remote location are provided, wherein the patient is onboard a mobile platform such as a commercial aircraft, and the vital health statistics are transmitted to, for example, an emergency treatment center during an in-flight emergency. The vital health statistics are preferably transmitted to the remote location via the Internet, although other transmission mediums such as cellular transmission may also be employed. The vital health statistics are gathered using a sensor that is in communication with the patient and which is also in communication with a signal processing electronics. The signal processing electronics transmit the statistics to a computing device such as a laptop computer or a personal digital assistant (PDA), among others. The computing device and/or an onboard server then transmits the statistics to the remote location.

### CROSS-REFERENCE TO RELATED APPLICATIONS

The present application is a continuation in part of U.S. application Ser. No. 09/952,612 titled “Method For Transmitting Vital Health Statistics to a Remote Location From an Aircraft,” filed Sep. 13, 2001 and presently pending.

### FIELD OF THE INVENTION

The present invention relates generally to mobile communication systems, and more particularly, to systems that transmit vital health statistics from a patient onboard a mobile platform to a remote location.

### BACKGROUND OF THE INVENTION

Telemedicine is an emerging field that generally relates to medical care access for consumers and health professionals via telecommunications technologies. One application of telemedicine involves the remote monitoring of vital health statistics of a patient and transmitting the statistics to a healthcare facility such as a hospital or medical clinic. For example, a patient may measure blood pressure or blood glucose levels and transmit the measurements to their healthcare provider so that the patient can be monitored more frequently and without physical visits to an office or healthcare facility. Additionally, devices are known that are used by the patient and healthcare provider to monitor a variety of vital health statistics, however, the devices can be relatively expensive, large, and heavy.

For example, U.S. Pat. No. 5,997,476 to Brown discloses a networked system for interactive communication and remote monitoring of patients, wherein a monitoring device is provided that produces measurements of physiological conditions of the patient, such as blood glucose, and records the measurements for transmission from a remotely programmable apparatus to a system server. The remotely programmable apparatus in one form is a personal computer or remote terminal connected to the server via a wide area network such as the Internet. Unfortunately, the system also requires the patient to answer a variety of questions through a user interface on a computer, which are transmitted from the system server, which may not be practical in certain situations, e.g. a medical emergency.

A further known telemedicine device is disclosed in U.S. Pat. No. 6,113,540 to Iliff, wherein both diagnostic and treatment advice is provided when a user (patient) accesses a system over a telephone network. Software algorithms provide diagnostic and treatment information based on inputs (complaints) from the patient. However, the system of Iliff does not include a means for measuring vital health statistics and transmitting the statistics to a remote location in real time. Further, the system of Iliff is relatively large and heavy and is not designed for a mobile patient.

Although known telemedicine devices that transmit vital health statistics to a remote location are effective in measuring and transmitting the appropriate information, no device has yet been developed for efficient use on a mobile platform. For example, cases of in-flight medical emergencies sometimes occur on commercial flights, wherein a passenger or crew member is critically ill and an emergency treatment team or center is waiting on the ground for the arrival of the flight in order to treat the patient. Unfortunately, use of telemedicine devices of the known art would not be possible due to the lack of a communications medium onboard the flight. Further, the many known telemedicine devices would be cost, space, and weight prohibitive for use on an aircraft.

Accordingly, there remains a need in the art for a telemedicine system and method that can measure and transmit vital health statistics from a patient onboard a mobile platform, e.g. a commercial aircraft, to a remote location, such as an emergency treatment center. A further need exists for a telemedicine system that is relatively compact, lightweight and inexpensive, and which is compatible with commonly used computing devices such as laptop computers and personal digital assistants (PDAs).

### SUMMARY OF THE INVENTION

In one preferred form, the present invention provides a method of providing vital health statistics to a remote location from a mobile platform comprising the steps of measuring at least one vital health statistic, transmitting the vital health statistic to signal processing electronics, transmitting the vital health statistic from the signal processing electronics to a computing device on a mobile platform, and transmitting the vital health statistic to the remote location. In another form, the vital health statistic is transmitted from the signal processing electronics to an onboard server, wherein the onboard server then transmits the vital health statistic to the remote location. Further, the computing device may transmit the vital health statistic to an onboard server, wherein the onboard server then transmits the vital health statistic to the remote location.

In another form, a system for transmitting vital health statistics to a remote location from a mobile platform is provided. The system comprises at least one sensor in communication with a patient, signal processing electronics in communication with the sensor, a computing device in communication with the signal processing electronics, and a mobile communications system. In operation, the sensor receives at least one vital health statistic from the patient and transmits the vital health statistic to the signal processing electronics, the signal processing electronics transmit the vital health statistic to the computing device, and the computing device transmits the vital health statistic to the remote location via the mobile communications system. In another form, the system comprises an onboard server rather than a computing device, wherein the onboard server receives the vital health statistic from the signal processing electronics and transmits the vital health statistic to the remote location. Alternately, the system comprises a computing device and an onboard server, wherein the computing device transmits the vital health statistic to the onboard server, and the onboard server transmits the vital health statistic to the remote location as previously described.

Preferably, the sensor is secured to the patient using a wrist cuff, for example, comprising hook and loop fasteners. Additionally, the sensor may be secured to the patient using a finger sensor unit. Further, the sensor is in communication with signal processing electronics, which are disposed adjacent the wrist cuff or finger sensor unit. The signal processing electronics are is communication the computing device, such as a personal computer, a laptop, or a personal digital assistant (PDA), among others, using a hard-wired connection. Alternately, the sensor may communicate with the computing device wirelessly using, for example, optical transmission or radio frequency (RF) transmission, among other communication mediums.

In one form of the present invention, the vital health statistics are transmitted to the remote location via a wide area network, for example, the Internet, wherein the mobile platform transmits and receives data to and from the remote location. Alternately, the vital health statistics may be transmitted to the remote location using cellular communications or other communications links established between the mobile platform and the remote location.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a diagram illustrating communication of vital health statistics between mobile platforms and remote locations in accordance with the present invention;

FIG. 2 is a diagram illustrating a system for transmitting health statistics to a remote location from a mobile platform; and

FIG. 3 is an illustration of a wrist cuff and a finger sensor unit constructed in accordance with the teachings of the present invention.

### DETAILED DESCRIPTION OF THE PREFERRED EMBODIMENTS

The following description of the preferred embodiments is merely exemplary in nature and is in no way intended to limit the invention, its application, or uses.

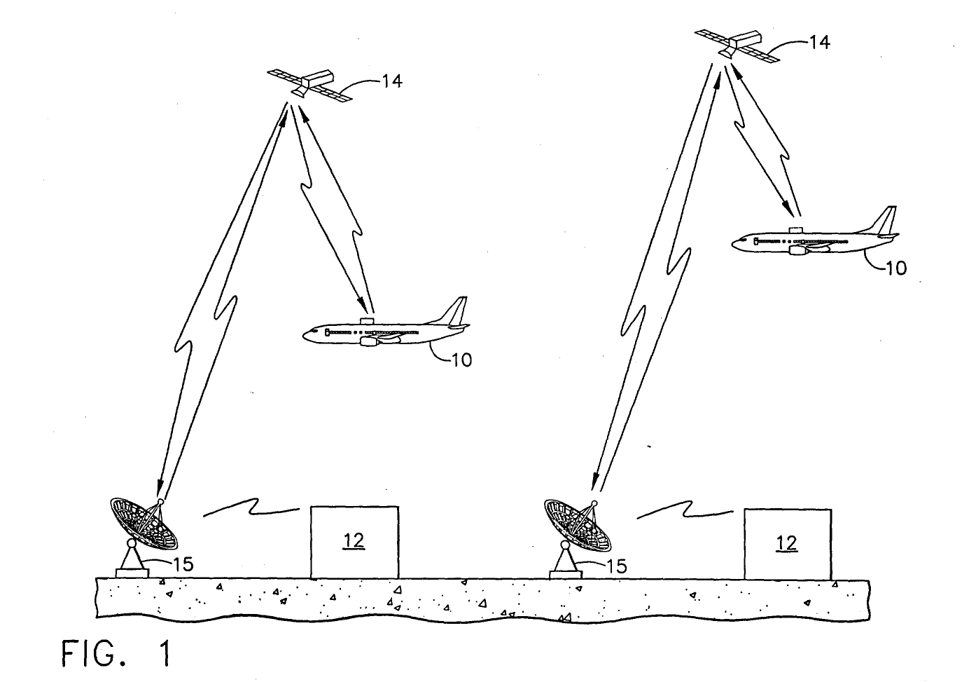

Referring to FIG. 1 of the drawings, a system for transmitting vital health statistics to a remote location from a mobile platform according to the present invention is facilitated through communications between a mobile platform 10, e.g., a commercial aircraft, and a remote location 12, e.g., emergency center, hospital, as shown in FIG. 1. Generally, the mobile platform 10 communicates, i.e. receives and transmits data, with a remote location 12 through data links with a satellite 14 and a ground station 15, or a plurality thereof. Further, the ground station 15 transmits the vital health statistics to the remote location 12 either through wireless or hard-wired communications as commonly known in the art. Accordingly, data is being transmitted between the mobile platform 10 and the remote location 12 as shown, which is hereinafter referred to as a mobile communications system.

Although the detailed description herein is directed to a system wherein the mobile platform is an aircraft, the invention is also applicable to other modes of mass transit such as ship, train, bus, and others, and the reference to aircraft should not be construed as limiting the scope of the present invention. Accordingly, an aircraft is also referred to as a mobile platform to encompass other modes of transportation to which the present invention may be applied.

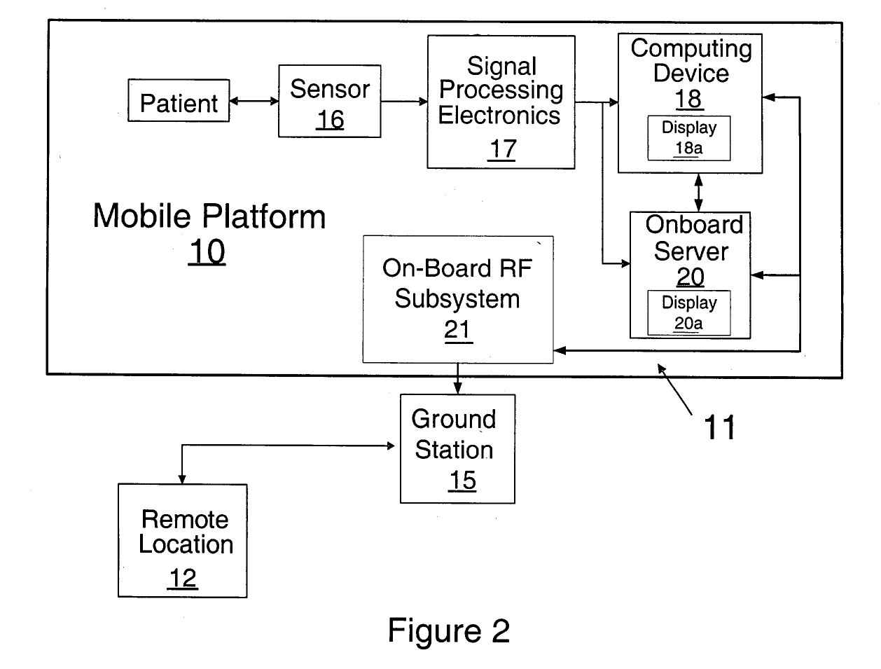

Referring now to FIG. 2, a system for transmitting vital health statistics to a remote location 12 from a mobile platform 10 is illustrated and generally indicated by reference numeral 11. The system comprises a sensor 16 that measures vital health statistics of a patient, such as blood pressure, pulse, body temperature and heart rate, and transmits these statistics to signal processing electronics 17 as shown. In one form, the signal processing electronics 17 comprise an analog-to-digital (A/D) converter to convert analog signals from the sensor 16 into digital signals. As further shown, the signal processing electronics 17 are in communication with a computing subsystem, namely a computing device 18 and/or an onboard server 20, wherein the digital signals are transmitted from the signal processing electronics 17 to the computing device 18 and/or the onboard server 20. The computing device 18 and/or the onboard server 20 transmits the vital health statistics in real time to a radio frequency (RF) communications subsystem 21 carried by the mobile platform 10 which in turn relays the information to the remote location 12 via the ground station 15.

In operation, the computing device 18 and/or the onboard server 20 transmit the vital health statistics to the remote location 12, e.g. an emergency treatment center, real time during an in-flight emergency or a heightened medical situation while in transit. Accordingly, the remote location 12 is continuously updated as to the status of the patient so that proper medical attention may be administered upon arrival of the flight. Furthermore, the remote location 12 may transmit data back to the computing device 18 and/or the onboard server 20 as necessary.

The computing device 18 preferably comprises a user interface that includes, but is not limited to, a sensor data display 18a, patient information, patient condition, and quality of the communications link. Furthermore, graphic indicators are provided on the user interface to indicate information such as communication quality, communication link established, data quality, and sensor operation. Moreover, interaction with the computing device 18 is preferably accomplished through a keyboard and/or pointing device(s), such as a computer mouse, in communication with the computing device 18, and patient interaction is not specifically required according to the system and method of the present invention.

The onboard server 20 also further comprises a display 20a. The display indicates information such as communication quality, communication link established, data quality, and sensor operation. The display may be any type known in the art and may comprise one or a combination of a CRT, liquid crystal display (LCD) panel or light emitting diodes (LEDs).

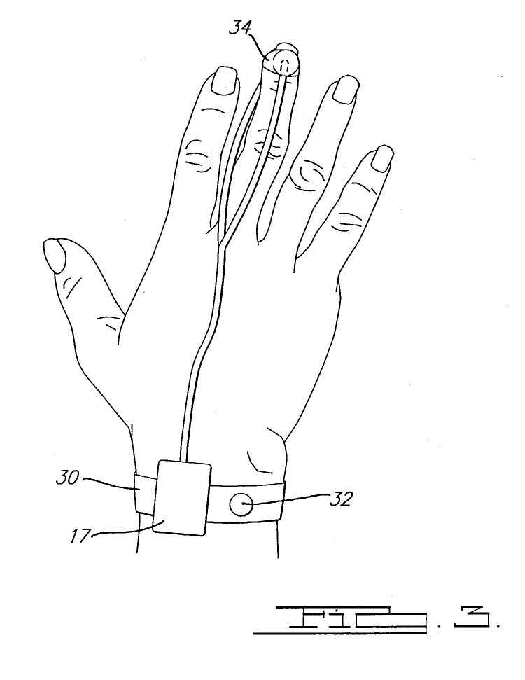

Referring now to FIG. 3, the signal processing electronics 17 in one form are attached to a wrist cuff 30 that is preferably a nylon web material wrapped around the wrist of a patient and secured using hook and loop fasteners. The wrist cuff may include, by way of example, a blood pressure sensor 32 as the sensor 16. In another form, a finger sensor unit 34 may be disposed adjacent the wrist cuff 30 and the signal processing electronics 17, wherein oxygen level monitoring may be accomplished, among other types of monitoring for vital health statistics. One type of suitable finger sensor 34 is the Universal Probe UD-5C manufactured by Minolta Corporation, however any other type of pulse oximeter may be employed. Preferably, the signal processing electronics 17 are encased in a sealed, shock-resistant semi-rigid container.

Since the sensor 16 is preferably secured to the patient using a wrist cuff 30 and/or a finger sensor 34 as described, the sensor 16 may be easily secured to and removed from the patient as necessary. Alternately, other devices and fasteners may also be employed in accordance with the teachings of the present invention, such as flexible fabric bands with elastic securement means, among others commonly known in the art.

The sensor 16 is preferably capable of monitoring vital health statistics such as EKG (electrocardiograph), temperature, pulse rate, blood oxygen level, blood pressure and blood glucose, among others commonly known in the art. The vital health statistics as disclosed herein are merely exemplary and shall not be construed as limiting the scope of the present invention.

As shown, the signal processing electronics 17 are further in communication with the computing device 18, which may be a personal computer, a laptop computer, or a personal digital assistant (PDA), among others. In one form, the signal processing electronics 17 communicate with the computing device 18 through a hard-wired connection, which may be a USB (universal serial bus) or SCSI (small computer system interface) cable, wherein the USB preferably powers the sensor 16. In another form, the signal processing electronics 17 communicate with the computing device 18 through wireless communication, such as optical or radio frequency (RF) transmission. As such, the signal processing electronics 17 would further comprise a battery source (not shown) for the necessary power. Accordingly, the system 11 according to the present invention is relatively lightweight and compact, and is thus suitable for use on a mobile platform such as a commercial aircraft. Furthermore, the sensor 16 and the signal processing electronics 17 may be provided as a stand-alone unit for use with a plurality of computing platforms.

Alternately, the sensor 16 may be in direct communication with the onboard server 20 through communications ports installed throughout the aircraft, for example, at selective passenger seats, rather than through the computing device 18. The sensor 16 remains secured to the patient to monitor the vital health statistics and is engaged with a communication port to transmit the statistics to the onboard server 20, which then transmits the statistics to the remote location 12 as previously set forth.

In another form of the present invention, the vital health statistics are transmitted to the remote location 12 from the mobile platform 10 via a wide area network such as the Internet. Accordingly, healthcare professionals at the remote location 12 access a web site to monitor the vital health statistics during the flight. Alternately, the vital health statistics may be transmitted to the remote location 12 via cellular communications, or through other communications mediums available onboard the mobile platform 10.

In operation, the sensor 16 is secured to the patient proximate the area where vital health statistics are to be monitored. For example, the sensor 16 may be placed around a wrist of the patient, preferably with a cuff comprising hook and loop fasteners, to monitor pulse. The pulse information is then transmitted from the sensor 16 to the signal processing electronics 17. The signal processing electronics 17 then transmit the pulse information to the computing device 18, which may then transmit the pulse information either to the onboard server 20 or directly to the remote location 12 via a cellular phone link. If the computing device 18 transmits the pulse to the onboard server 20, the onboard server 20 then transmits the pulse to the remote location 12 via the radio frequency communications subsystem 21.

Further, healthcare professionals at the remote location 12, which may be an emergency treatment center, for example, receive the pulse information and plan medical treatment as necessary. The healthcare professionals may advise onboard personnel how to treat the patient until the aircraft lands, in addition to acquiring the necessary medical equipment and staff to treat the patient upon arrival. The healthcare professionals may be monitoring the vital health statistics via the Internet through access to a web page, which may further be secured in order to maintain the confidentiality of the information relating to the patient. Alternately, the vital health statistics may be received via cellular transmissions from the mobile platform 10.

The system according to the present invention is preferably compatible with other telemedicine devices by way of IEEE 1516 specification in conjunction with the Health Level 7 and American Telemedicine Association (HLA/HL7) protocol. Preferably, the system of the present invention is compatible with telemedicine services known in the art such as MedLinke from MedAire and CyberCare® 24 from CyberCare. Furthermore, the system of the present invention is compatible with FAA (Federal Aviation Administration) requirements on portable electronic devices that do no cause electromagnetic interference (EMI) with aircraft systems.

Accordingly, a system and method for transmitting vital health statistics to a remote location from a mobile platform is provided for an increased level of healthcare during transit. The system is further compact, lightweight, and less expensive than devices of the known art and may transmit the vital health statistics using a variety of communication mediums from a mobile platform to a remote location.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A method of providing vital health statistics to a remote location from a mobile platform, the method comprising the steps of an individual on-board the mobile platform:
(a) measuring at least one vital health statistic;
(b) transmitting the vital health statistic to a computing subsystem on a mobile platform; and
(c) transmitting the vital health statistic from the computing subsystem to the remote location.

2. The method of claim 1 further comprising the steps of:
(a) transmitting the vital health statistic from the computing subsystem to an onboard server on the mobile platform; and
(b) transmitting the vital health statistic from the onboard server to the remote location.

3. The method of claim 1, wherein the vital health statistic is measured using a sensor in communication with the computing subsystem and a patient.

4. The method of claim 3, wherein the sensor is secured to the patient with a wrist cuff comprising hook and loop fasteners.

5. The method of claim 3, wherein the sensor communicates with the computing device through a hard-wired connection.

6. The method of claim 3, wherein the sensor communicates with the computing device through a wireless connection.

7. The method of claim 1, wherein the vital health statistic is transmitted to the remote location via a wide area network.

8. The method of claim 1, wherein the vital health statistic is transmitted to the remote location via cellular transmission.

9. A method of providing vital health statistics to a remote location from a mobile platform, the method comprising the steps of an individual on-board the mobile platform:
(a) measuring at least one vital health statistic;
(b) transmitting the vital health statistic to signal processing electronics;
(c) transmitting the vital health statistic from the signal processing electronics to an onboard server on a mobile platform; and
(d) transmitting the vital health statistic from the onboard server to the remote location.

10. The method of claim 9, wherein the vital health statistic is measured using a sensor in communication with a computing device and a patient.

11. The method of claim 9, wherein the vital health statistic is transmitted to the remote location via a wide area network.

12. The method of claim 9, wherein the vital health statistic is transmitted to the remote location via cellular transmission.

13. A method of providing vital health statistics to a remote location from a mobile platform, the method comprising the steps of the steps of an individual on-board the mobile platform:
(a) measuring at least one vital health statistic;
(b) transmitting the vital health statistic to signal processing electronics;
(c) transmitting the vital health statistic from the signal processing electronics to a computing device on a mobile platform;
(d) transmitting the vital health statistic from the computing device to an onboard server on the mobile platform; and
(e) transmitting the vital health statistic from the onboard server to the remote location.

14. The method of claim 13, wherein the vital health statistic is measured using a sensor in communication with the computing device and a patient.

15. The method of claim 13, wherein the vital health statistic is transmitted to the remote location via a wide area network.

16. The method of claim 13, wherein the vital health statistic is transmitted to the remote location via cellular transmission.

17. A system for transmitting vital health statistics to a remote location from a mobile platform comprising:
at least one sensor in communication with a patient;
signal processing electronics in communication with the sensor;
a computing device in communication with the signal processing electronics; and
a mobile communications system,
wherein the sensor receives at least one vital health statistic from the patient and transmits the vital health statistic to the signal processing electronics, the signal processing electronics transmit the vital health statistic to the computing device, and the computing device transmits the vital health statistic to the remote location via the mobile communications system.

18. The system of claim 17 further comprising a wrist cuff, wherein the sensor is disposed proximate the patient using the wrist cuff.

19. The system of claim 18, wherein the wrist cuff comprises hook and loop fasteners.

20. The system of claim 18 further comprising a finger sensor unit disposed proximate the wrist cuff and in communication with the signal processing electronics.

21. The system of claim 17, wherein the computing device comprises a personal computer.

22. The system of claim 17, wherein the computing device comprises a personal digital assistant.

23. The system of claim 17, wherein the computing device comprises a laptop computer.

24. The system of claim 17, wherein the signal processing electronics further comprise an analog to digital converter.

25. A system for transmitting vital health statistics to a remote location from a mobile platform comprising:
at least one sensor in communication with a patient;
signal processing electronics in communication with the sensor;
an onboard server on the mobile platform in communication with the signal processing electronics; and
a mobile communications system,
wherein the sensor receives at least one vital health statistic from the patient and transmits the vital health statistic to the signal processing electronics, the signal processing electronics transmit the vital health statistic to the onboard server, and the onboard server transmits the vital health statistic to the remote location via the mobile communications system.

26. The system of claim 25 further comprising a wrist cuff, wherein the sensor is disposed proximate the patient using the wrist cuff.

27. The system of claim 26, wherein the wrist cuff comprises hook and loop fasteners.

28. The system of claim 26 further comprising a finger sensor unit disposed proximate the wrist cuff and in communication with the signal processing electronics.

29. The system of claim 25, wherein the signal processing electronics further comprise an analog to digital converter.

30. A system for transmitting vital health statistics to a remote location from a mobile platform comprising:
at least one sensor in communication with a patient;
signal processing electronics in communication with the sensor;
a computing device in communication with the signal processing electronics;
an onboard server in communication with the computing device; and
a mobile communications system,
wherein the sensor receives at least one vital health statistic from the patient and transmits the vital health statistic to the signal processing electronics, the signal processing electronics transmit the vital health statistic to the computing device, the computing device transmits the vital health statistic to the onboard server, and the onboard server transmits the vital health statistic to the remote location via the mobile communications system.

31. The system of claim 30 further comprising a wrist cuff, wherein the sensor is disposed proximate the patient using the wrist cuff.

32. The system of claim 31 further comprising a finger sensor unit disposed proximate the wrist cuff and in communication with the signal processing electronics.

33. The system of claim 30, wherein the computing device comprises a personal computer.

34. The system of claim 30, wherein the computing device comprises a personal digital assistant.

35. The system of claim 30, wherein the computing device comprises a laptop computer.

36. The system of claim 30, wherein the signal processing electronics further comprise an analog to digital converter.
